(19) United States
(12) Patent Application Publication
(10) Pub. No.: US 2005/0085754 A1
(43) Pub. Date: Apr. 21, 2005

(54) Device for externally immobilizing broken bones

(76) Inventors: Gerd Werding, Ingolstadt (DE); Willi Schneider, Bad Neustadt a.d. Saale (DE)

Correspondence Address: NIXON PEABODY, LLP, 401 9TH STREET, NW, SUITE 900, WASHINGTON, DC, 20004-2128, US

(21) Appl. No.: 10/504,094

(22) PCT Filed: Feb. 10, 2003
(86) PCT No.: PCT/EP03/01293

(30) Foreign Application Priority Data
Feb. 11, 2002 (DE) 20202049.5

(51) Int. Cl. A61F 5/00

(52) U.S. Cl. 602/21

(57) ABSTRACT

The invention relates to a device for externally immobilizing broken bones, particularly bones of the extremities. According to the invention, the device has a multi-articulartion rod that can be place don the outside of the body while extending along a broken bone. This rod comprises elements (1), which can move in relation to one another and can be fixed in appointed directions. One of the elements (1) has a bone pin (7), which can be held therein in a manner that permits it to turn and which is provided for being anchored inside the bone. In addition, the tip of at least one of the bone pins (7) is provided in the form of a screw having a self-tapping thread (8).

The invention concerns a device of the type referred to in the preamble to claim 1 for externally immobilizing broken bones.

DE 19746687 A1 discloses a device of this type. The bone pins of the known device feature smooth tips, whereby the pins held in the elements and turnable in them are embedded into the bones using a small electric drill, and under particular hand-applied pressure. The embedded bone pin is then fixed to the element of the device to which it belongs by means of a counternut tightened against the element.

The disadvantage of the known device is that embedding the bone pin involves relatively high equipment use, because in the process of being secured the bone pin must be embedded relatively deeply into the bone because of the smoothness of its tip, and because the use of the electric drill for the purpose of embedding the pin causes unnecessary stress to the bone through the high rotational speed that is used and also required in this procedure.

Therefore the challenge of this invention is to improve a device of the type mentioned above in such a way that the bone pin can be anchored securely with the least possible stress to the bone.

According to the invention this challenge is solved through a device for externally immobilizing broken bones with the distihguishing features of claim 1.

The achievement resulting from the bone pin of this design will be that, using a tool such as a spanner or screwdriver, the pin can be screwed into the bone, and that it will in this manner become anchored in a way similar to that of a corkscrew being screwed into a cork. Depths of penetration that are substantially lower than those used for known bone pins will be adequate for securely anchoring the bone pins to the bones.

The preferred embodiment of the invention in accordance with claim 2 achieves the result that the screwed in bone pin remains in its screwed in position, thereby preventing it from unscrewing itself loose from the bone independently.

In the preferred embodiment of the invention in accordance with claim 3, it is possible to adjust the height of the device's element holding the bone pin in relation to the bone once the pin has been screwed in.

A preferred embodiment of the invention is described as illustrated in the attached drawings. In these:

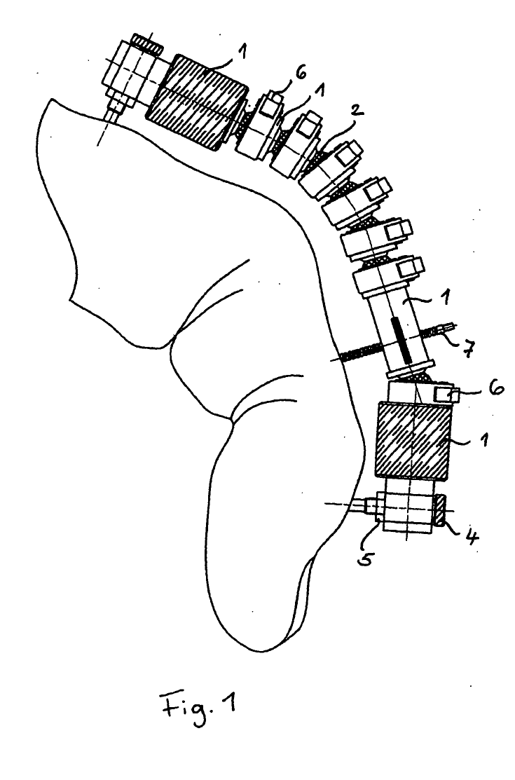

FIG. 1 illustrates a device for externally immobilizing bones used on a finger according to the invention, and

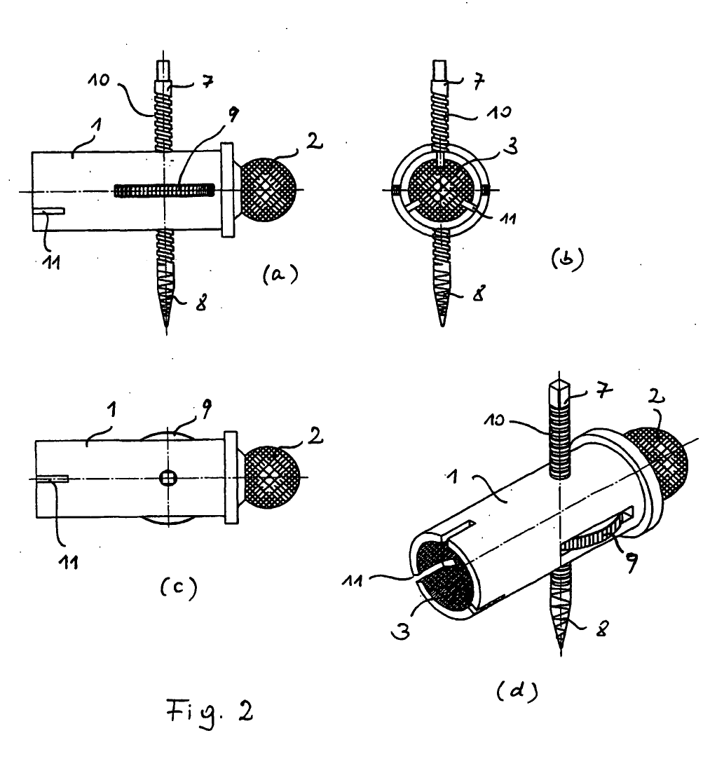

FIG. 2(a)-(d) illustrates an element of the device which holds a bone pin that can be screwed into the bone. The element is shown in a side view, rear view, from above and in perspective.

As can be seen from FIG. 1, the immobilization device features a chain of elements 1 which, as can be seen more clearly in FIG. 2, are joined to one another in ball joint fashion. Each element features a ball on one end and a ball socket 3 on the other, whereby each ball 2 of one element is contained by ball socket 3 of the adjacent element. The relative position of the elements that are joined in this manner in a ball and socket style is established by the clamps 6 that enclose the ball socket, pressing together ball sockets 3 that have been given elastic deformation capabilities by the provision of slits 11, against the balls 2 which they hold.

At the ends of the immobilization device there are conventional type bone pins that can be grasped at the head 4 with an electric drill by way of example. After having been inserted into the bone, the bone pin can be screwed tight against the element in which it is located with a counternut.

Bone pin 7, in accordance with the invention, is situated in an element located between the ends. This constitutes a screw featuring a tip having a self-tapping thread 8, similar to a self-tapping screw or a corkscrew. When it is screwed in, the bone pin becomes anchored to the bone. In this way stability is achieved at a smaller penetration depth than is the case with conventional bone pins, as illustrated at the ends of the immobilization device.

Bone pin 7 is fixed to element 1 through a knurled nut 9 that is housed in such a way that it can be turned but cannot be displaced axially. By means of the knurled nut 9 the bone pin 7 works together with a self-locking thread 10. If the knurled nut 9, which is accessible from the outside (insofar as it is needed), is held with the hand and the screw is turned at the head, either using a tool or by hand, then the bone pin 7 moves in the bone. The length of the portion having the self-locking thread 10 above the element 1 approximately corresponds to the length of the of the portion having the self-threading thread 8 from its tip to the element 1. The result achieved by the self-locking action is that the bone pin 7 is safe against unwanted turning without need for any further measures.

If, after it has been screwed into the bone, the bone pin 7 is held, and the knurled nut 9 is turned, then the height of the element holding bone pin 7 can be changed in relationship to the bone. The thread's self-locking nature also ensures that once the position has been established, it will not change by itself.

In the illustrated embodiment only the bone pin situated in the middle has been developed in accordance with the invention, while the bone pins at either end are of the conventional type. It is also possible, of course, to provide all bone pins of the same type as the middle bone pin illustrated in FIG. 1.

1. A device for externally immobilizing broken bones, particularly bones of the extremities, comprising a multi-articulation rod that can be placed on the outside of the body while extending along a broken bone, said rod comprising elements (1) which can move in relation to one another and can be fixed in desired directions with respect to one another, one of the elements (1) having a bone pin (7) which can be held therein in a manner that permits it to turn and which has means for being anchored inside the bone, characterized in that the tip of at least one of the bone pins (7) is in the form of a screw having a self-tapping thread (8).

2. A device as claimed in claim 1, characterized in that said bone pin (7) having a tip in the form of said self-tapping thread also has along its shaft a self-locking thread (10) extending into the element (1) associated with it, the length of the portion of the bone pin (7) from the thread at the tip of the self-threading portion to such element and the length of the portion of of self locking thread extending from said element being approximately the same.

3. A device as claimed in claim 2, characterized in that, for inserting the shaft of the bone pin into element (1) a housed knurled nut (9) is provided that is accessible to the hand and can be turned in the element but cannot be moved axially out of place with respect to element (1) the self-locking thread (10) of the bone pin (7) engaging and being held by the knurled nut.
